(19) United States
(12) Patent Application Publication
(10) Pub. No.: US 2026/0021305 A1
(43) Pub. Date: Jan. 22, 2026

(54) RECORDING AND RECOVERY OF SIGNALS DURING STIMULATION

(71) Applicant: EMORY UNIVERSITY, Atlanta, GA (US)

(72) Inventors: Enrico OPRI, Atlanta, GA (US); Svjetlana MIOCINOVIC, Atlanta, GA (US)

(21) Appl. No.: 18/993,727

(22) PCT Filed: Jul. 21, 2023
(86) PCT No.: PCT/US2023/028391

§ 371 Date: Jan. 13, 2025

Related U.S. Application Data
(60) Provisional application No. 63/391,562, filed on Jul. 22, 2022.

(51) Int. Cl. A61N 1/36; A61N 1/05

(52) U.S. Cl. CPC A61N 1/36139; A61N 1/0534; A61N 1/36125

(57) ABSTRACT

Described herein are systems and devices for recording a signal at a stimulating electrode. An example device includes a stimulator port operably connected to a stimulator decoupler stage; an input port operably connected to the stimulator decoupler stage and a current limiter stage, where the current limiter stage is configured to limit a current flowing from the input port to a recording port and where the stimulator decoupler stage is configured to prevent a loading effect on the recording amplifier inputs; a voltage range limiter stage operably connected to the current limiter stage and the recording port and configured to limit the voltage output to a predetermined recording amplifier range.

### CROSS-REFERENCE TO RELATED APPLICATIONS

This application claims the benefit of U.S. provisional patent application No. 63/391,562, filed on Jul. 22, 2022, and titled “RECORDING AND RECOVERY OF SIGNALS DURING STIMULATION,” the disclosure of which is expressly incorporated herein by reference in its entirety.

### STATEMENT REGARDING FEDERALLY FUNDED RESEARCH

This invention was made with government support under grant/contract number K23NS097576 awarded by the National Institute of Health, grant/contract number 1P50NS098685 awarded by the National Institute of Health, and grant/contract number T32NS007480-20 awarded by the National Institute of Health. The government has certain rights in the invention.

### BACKGROUND

Deep Brain Stimulation (DBS) is a surgical therapy that can be used to treat Parkinson's disease, essential tremor, dystonia, and other neuropsychiatric disorders. DBS can include an implanted pulse generator that delivers electrical pulses to DBS-type electrodes implanted in a specific brain region of interest, for therapeutic benefit. Oscillatory and stimulation evoked neural activity correlates with pathophysiology and spatial localization of the brain targets used for DBS, and their understanding can lead to further benefits and therapy improvements for the affected patients.

However, recovering viable electrophysiology, and specifically, neural activity after each single stimulation pulse, is non-trivial in spatially constrained electrodes, especially if recording and stimulating from the same electrodes. This can be due to saturation and high differential input voltage at the amplifier/pre-amplifier stage.

Therefore, what is needed are systems and devices configured to allow for simultaneous recording and simulation with one or more electrodes.

### SUMMARY

In some aspects, the techniques described herein relate to a device for recording a signal at a stimulating electrode, the device including: a stimulator port operably connected to a stimulator decoupler stage; an input port operably connected to the stimulator decoupler stage and a current limiter stage, wherein the current limiter stage is configured to limit a current flowing from the input port to a recording port and wherein the stimulator decoupler stage is configured to prevent a loading effect on an input port of a recording amplifier; a voltage range limiter stage operably connected to the current limiter stage and the recording port and configured to limit a voltage output to a predetermined recording amplifier range.

In some aspects, the techniques described herein relate to a device, wherein the stimulator decoupler stage includes a pair of antiparallel diodes connected in parallel.

In some aspects, the techniques described herein relate to a device, wherein the stimulator decoupler stage includes a plurality of antiparallel diodes connected in series.

In some aspects, the techniques described herein relate to a device, wherein the current limiter stage includes a Junction Field Effect (JFET) constant current source, wherein the JFET constant current source includes a JFET transistor and a resistor.

In some aspects, the techniques described herein relate to a device, wherein the current limiter stage includes a plurality of JFET constant current sources.

In some aspects, the techniques described herein relate to a device, wherein the voltage range limiter stage includes a pair of antiparallel diodes connected in parallel.

In some aspects, the techniques described herein relate to a device, wherein the device further includes a ground port, and the voltage range limiter stage is operably connected between the ground port and the recording port.

In some aspects, the techniques described herein relate to a device, wherein the device is configured to receive an electrophysiology signal.

In some aspects, the techniques described herein relate to a device, wherein the device is configured amplify neural activity during deep brain stimulation.

In some aspects, the techniques described herein relate to a system for simultaneously stimulating and recording data using an electrode, the system including: a stimulator port operably connected to a stimulator decoupler stage; a ground port; an input port operably connected to the stimulator decoupler stage and a current limiter stage, wherein the current limiter stage is configured to limit a current flowing from the input port to a recording port and wherein the stimulator decoupler stage is configured to prevent a loading effect on an input port of a recording amplifier wherein the recording amplifier is operably connected to the recording port and the ground port; a probe including one or more electrodes operably connected to the input port; a voltage range limiter stage operably connected to the current limiter stage and the recording port and configured to limit a voltage output to a predetermined recording amplifier range; and a stimulator operably connected to the stimulator port.

In some aspects, the techniques described herein relate to a system, wherein the stimulator decoupler stage includes a pair of antiparallel diodes connected in parallel.

In some aspects, the techniques described herein relate to a system, wherein the stimulator decoupler stage includes a first pair of antiparallel diodes that are connected in parallel, and a second pair of antiparallel diodes connected in parallel, and wherein the first and second pair of antiparallel diodes are connected in series.

In some aspects, the techniques described herein relate to a system, wherein the current limiter stage includes a JFET constant current source, wherein the JFET constant current source includes a transistor and a resistor.

In some aspects, the techniques described herein relate to a system, wherein the current limiter stage includes a plurality of JFET constant current sources.

In some aspects, the techniques described herein relate to a system, wherein the voltage range limiter stage includes a pair of antiparallel diodes connected in parallel.

In some aspects, the techniques described herein relate to a system, wherein the system further includes a ground port, and the voltage range limiter stage is operably connected between the ground port and the recording port.

In some aspects, the techniques described herein relate to a system, wherein the stimulator and recording amplifier are a combined stimulator and recording amplifier.

In some aspects, the techniques described herein relate to a system wherein the stimulator, recording amplifier, voltage range limiter stage, current limiter stage, and stimulator decoupler stage are part of a single device.

In some aspects, the techniques described herein relate to a system, wherein the system is configured to receive an electrophysiology signal.

In some aspects, the techniques described herein relate to a system, wherein the system is configured amplify neural activity during deep brain stimulation.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1A illustrates a system block diagram of a stimulation and recording system, according to an example implementation of the present disclosure.

FIG. 1B illustrates a diagram of a system block diagram of an example implementation of the present disclosure.

FIG. 2A illustrates a circuit diagram of an example implementation of the present disclosure.

FIG. 2B illustrates a system block diagram of an example system for performing stimulation and recording, including a stimulator and recording amplifier, according to an example implementation of the present disclosure.

FIGS. 3A-3D illustrate a comparison of an example implementation of the present disclosure to an implementation including a Y-splitter. FIG. 3A illustrates a system block diagram of a Y splitter device for stimulation and recording. FIG. 3B illustrates an example implementation of the present disclosure for stimulation and recording. FIG. 3C illustrates a simulation of the current delivered to the patient and amplifier by the Y splitter device illustrated in FIG. 3A. FIG. 3D illustrates a simulation of the current delivered to the patient and recording amplifier by the example implementation of the present disclosure illustrated in FIG. 3B.

FIGS. 3E-3F illustrate additional examples of systems for performing stimulation and recording from the same electrode. FIG. 3E illustrates a system including a Y-splitter, and FIG. 3F illustrates a system including a switch.

FIG. 4 illustrates experimental results including an example implementation of the present disclosure.

FIG. 5 illustrates plots of signal distortion for an example implementation.

FIGS. 6A-6C illustrate example implementations of the present disclosure. FIG. 6A illustrates an example implementation used with a separate recording amplifier and stimulator. FIG. 6B illustrates an example implementation used with a combined recording amplifier and stimulator. FIG. 6C illustrates an example implementation combined with a recording amplifier and stimulator system.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. While implementations will be described for performing certain measurements (e.g. concentrations of pollutants), it will become evident to those skilled in the art that the implementations are not limited thereto, but are applicable to performing any kind of environmental measurement.

Implementations of the present application pertains generally to systems and methods for recording viable electrophysiology during stimulation delivery, and more particularly, but not by way of limitation, to systems and methods for recovering viable electrophysiological signals during delivery of deep brain stimulation (DBS).

Implementations of the present disclosure include systems that can deliver and record stimulation from the same or adjacent electrodes. A non-limiting application of delivering and recording stimulation from the same or adjacent electrodes is performing brain stimulation or DBS.

Implementations of the present disclosure include instrumentation for reducing stimulation artifacts at the acquisition stage of the amplifier. Reducing stimulation artifacts act the acquisition stage can be used to recover electrophysiologic signals during stimulation from the same or adjacent electrode contacts. This system and method can preserve oscillatory components within the electrophysiological signal, allowing the unaltered underlying activity after each stimulation pulse to be recorded.

Implementations of the present disclosure includes devices that can be configured for fully-passive clipping for recovering electrophysiology. Example implementations of the present disclosure are referred to herein as “FPClipre” (“fully passive clipping for recovering electrophysiology”). Implementations of the present disclosure can limit the differential input voltage at the amplifier stage and thereby avoid saturation and slow recovery from stimulation; enable linking separate stimulation and amplifier stages without requiring an active stage for switching, avoid a loading effect on the recording amplifier, ensure the delivery of the stimulator current to the electrode without unexpected loss through the recording amplifier, and allow for recording and stimulation to be performed using the same contact or electrode.

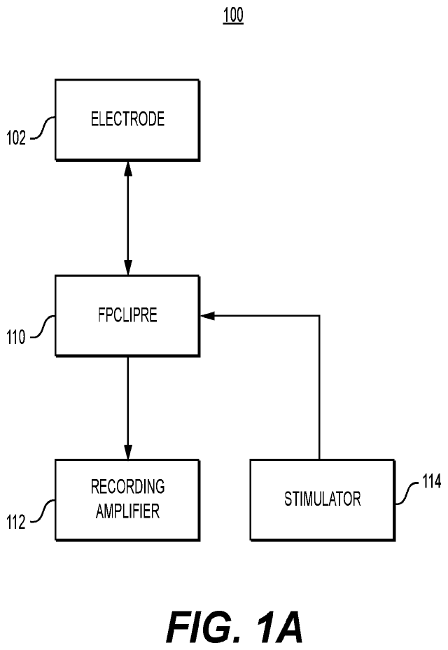

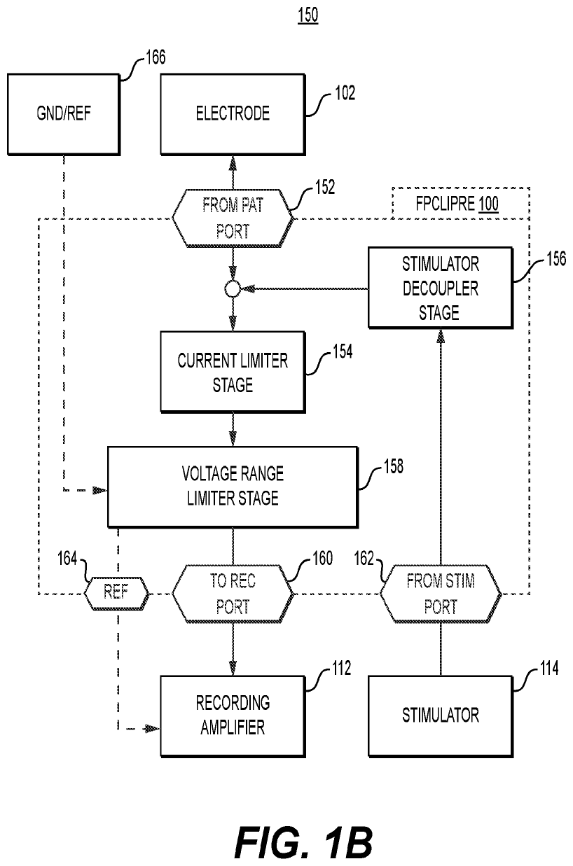

FIGS. 1A-1B illustrate block diagrams of an example implementation of the FPClipre in a recording and stimulation setup. FIG. 1A illustrates the main blocks/connections used in the example implementation to stimulate and record from the same electrode/contact.

With reference to FIG. 1A, a system block diagram is shown of a system 100 for performing stimulation and recording using the FPClipre 110. The system 100 can include an electrode 102, a recording amplifier 112, and a stimulator 114. The electrode 102 can be configured to receive stimulation from the stimulator 114 and also to transmit signals to the recording amplifier 112. The FPClipre device 110 is operably connected between the electrode 102 and the recording amplifier 112 and stimulator 114.

FIG. 1B illustrates a block diagram of the FPClipre instrumentation that can be used for reduction of stimulation artifact and enabling recording from the same contact used for stimulation. The FPClipre 110 is used in a system 150 including an electrode 102, a recording amplifier 112, and a stimulator 114. The FPClipre 110 can include an input port 152 configured to send/receive signals from the electrode 102. Implementations of the FPClipre device 110 can be configured as a hardware add-on in front of the amplifier 112 input and stimulation output of a stimulator 114.

The input and output (“I/O”) ports of the FPClipre 110 device can be “from Pat” 152 connected to patient electrode/contact; “from Stim” 162 connected to the stimulator output; “to Rec” 160 connected to the recording amplifier input; “REF” 164 connected to the reference (e.g. of a patient).

As shown in FIG. 1B, the FPClipre can include three stages. The “Current limiter stage” 154 can limit the current that can flow to the recording amplifier 112. Additionally, the current limiter stage 154 can ensure that most or all of the current delivered by the stimulator 114 is delivered to the patient by limiting the current that can flow to the recording amplifier 112.

The “Voltage range limiter stage” 158 can limit the voltage seen by the recording amplifier 112. The recording amplifier 112 can have a maximum input voltage that is lower than the voltage at the electrode 102, so the voltage range limiter stage 158 can prevent the voltage reaching the recording amplifier 112 from exceeding the maximum input voltage of the recording amplifier 112. If the maximum input voltage of the recording amplifier 112 is exceeded, it can cause saturation at the recording amplifier 112, and the recording amplifier 112 can be slow to recover from saturation. The “stimulator decoupler stage” 156 can prevent a loading effect on the recording amplifier 112.

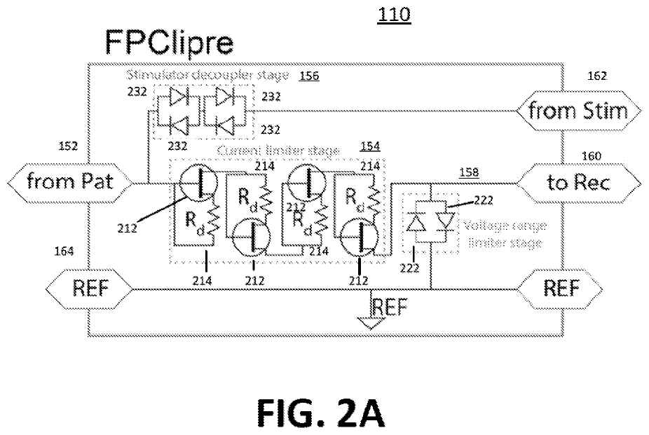

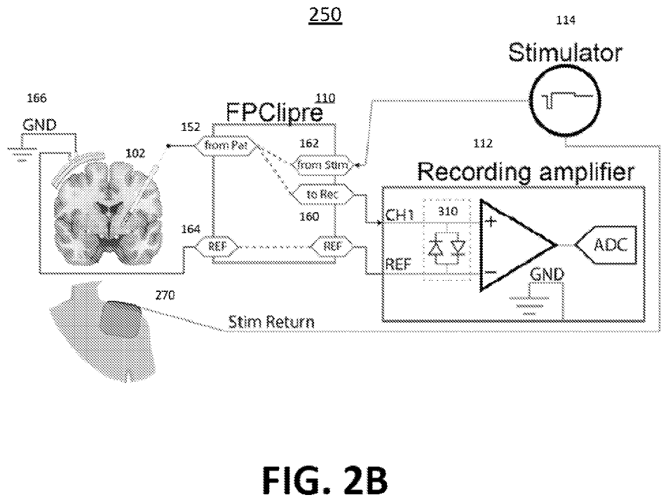

FIGS. 2A-2B illustrate diagrams of example implementations FPClipre instrumentation and application within a recording and stimulation setup. FIG. 2A illustrates a circuit diagram of an implementation of the FPClipre 110 that can be used to reduce stimulation artifacts and to enable recording from the same electrode contact used for stimulation. As described with reference to FIGS. 1A and 1B, the I/O ports of the device can include “from Pat” 152, which can be connected to patient electrode contact; “from Stim” 162, which can be connected to the stimulator output; “to Rec” 160, which can be connected to the recording amplifier input; and “REF” 164 which can be connected to the reference of the patient and recording amplifier 112.

Still with reference to FIG. 2A, the example FPClipre 110 can include three sections: a current limiter stage 154, a voltage range limiter stage 158, and a stimulator decoupler stage 156.

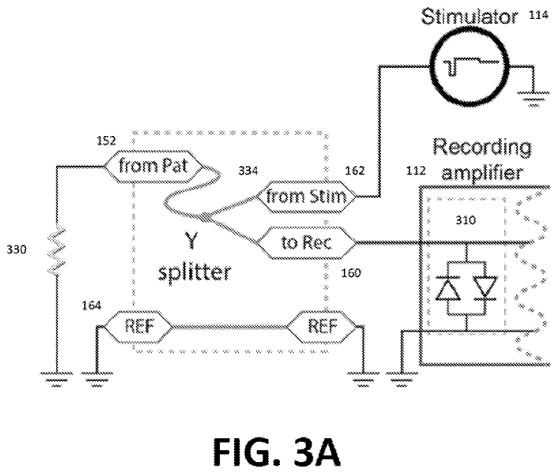

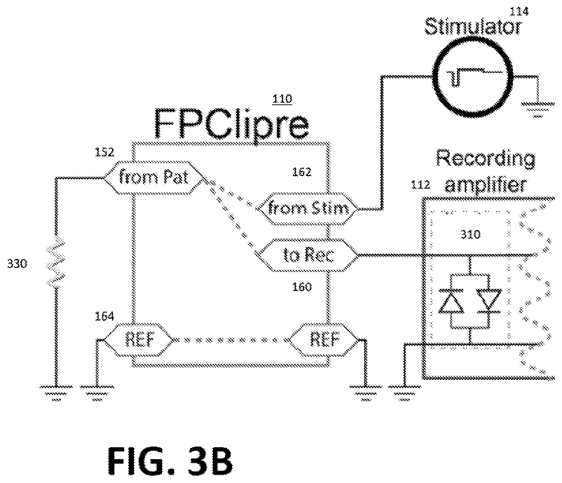

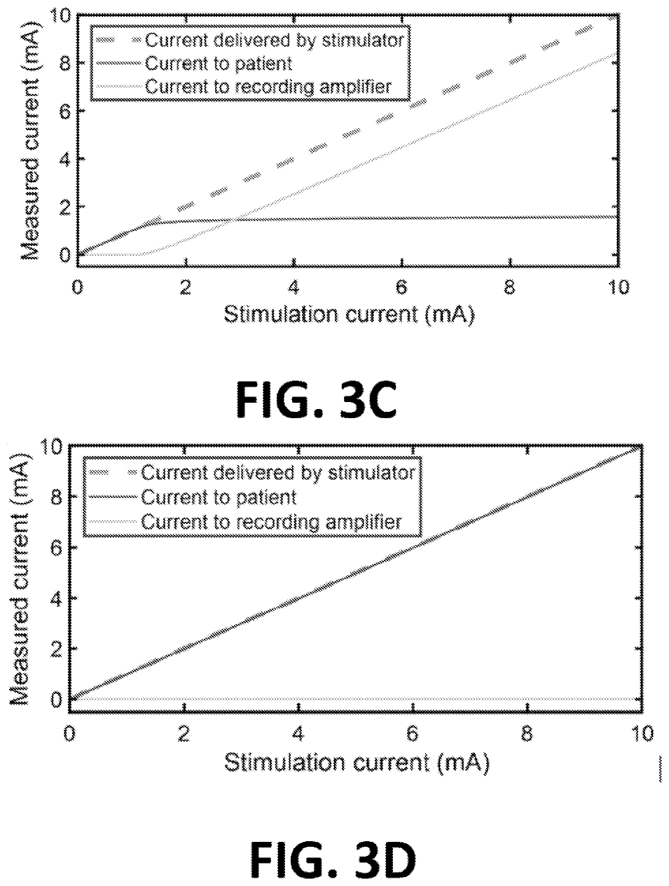

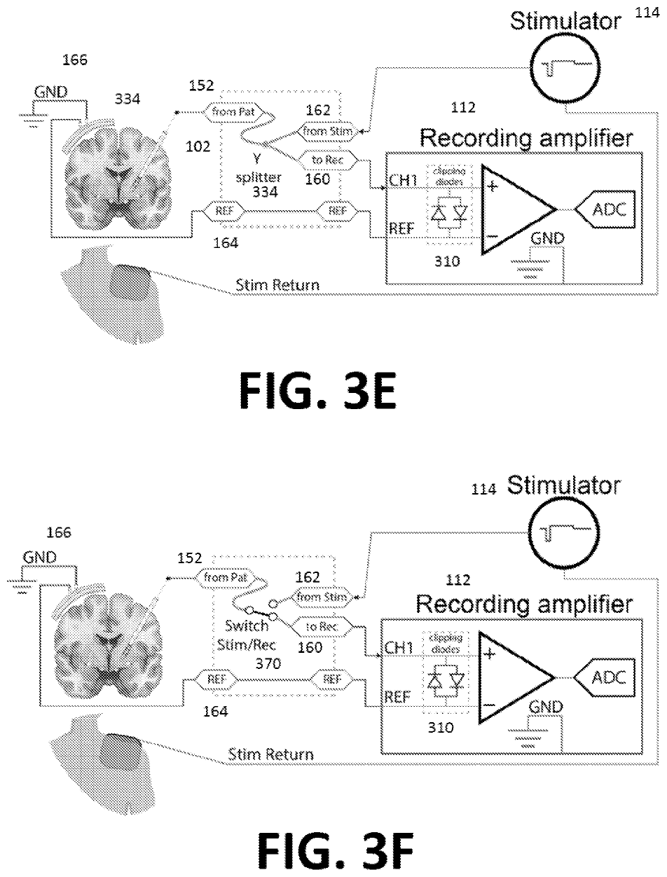

As shown in FIG. 2A, the current limiter stage 154 can be implemented with one or more pairs of junction-gate field-effect transistors (JFETs) 212 and resistors 214, where the pairs of JFETs 212 and resistors 214 can be linked in series. The JFETs 212 and resistors 214 can be selected to tune the current being limited. The “Current limiter stage” 154 can allow the current delivered to the stimulation port 162 to be delivered fully to the patient electrode 102, without the interference of the recording amplifier clipping diodes. FIGS. 3C-3D, described below, illustrate example results comparing the current delivered by the stimulator to the current delivered to the patient and the current delivered to the recording amplifier.

Still with reference to FIG. 2A, the voltage limiter stage 158 can limit the voltage output to the recording amplifier (shown as “to Rec” 160) in order to prevent saturation of the recording amplifier (not shown). The voltage limiter stage 158 can be configured to operate with the current limiter stage 154 and can be operably coupled to the output of the current limiter stage 154. As shown in FIG. 2A, the current limiter stage 154 is operably coupled to the “to Rec” 160 output of the device 110, and the voltage range limiter stage 158 is operably coupled between the “to Rec” port 160, the current limiter stage 154, and the ground 164. The voltage limiter stage 154 can be configured to avoid engaging the clipping diodes/stage of the connected recording amplifier (not shown), and can also be configured to engage the current limiting feature of the current limiter stage 154 without saturating the recording amplifier (not shown). Once saturated, the recording amplifier can have a slow recovery, which can reduce the quality of the recorded signals.

Still with reference to FIG. 2A, the voltage limiter stage 158 can be implemented using clipping diodes 222 as voltage range limiter stage 158. The clipping diodes 222 can be selected so that they become conductive before the recording amplifier clipping diodes/stage (not shown). The recording amplifier clipping diodes or clipping stage can be included in the recording amplifier as protection for the amplifier input. The voltage limiter stage 158 can be designed by choosing clipping diodes 222 (used for the voltage range limiter stage 158) with a lower forward voltage (V_{f}) than any clipping diodes in the recording amplifier. In addition, this allows implementations of the FPClipre 110 to be used with analog to digital converters (ADC) that do not have a high dynamic range, because the FPClipre can limit a stimulation artifact within the range specified by the clipping diode's V_{f} within the FPClipre 110, and the electrophysiology can be recoverable shortly after the ending of the stimulation pulse (e.g., <1 ms, tunable). Therefore, the voltage range limiter stage can avoid saturating the recording amplifier 112. In the example implementation shown in FIG. 2A, clipping diodes 222 are used for the voltage range limiter stage 158.

Implementations of the FPClipre device 110 can also include a “Stimulation decoupler stage” 156 that can include antiparallel diodes 232, which can be configured to prevent a loading effect on the recording amplifier inputs. The antiparallel diodes 232 can decouple the stimulator (not shown) so that it is not in parallel with the recording amplifier (not shown) (e.g. when the output of the stimulator is 0 mA or 0V). This can be used when the stimulator output impedance is much lower than the recording amplifier. A low stimulator output impedance in parallel with the recording amplifier (high impedance) can otherwise cause an increased voltage drop at the electrode contact impedance while decreasing the voltage drop at the recording amplifier input, consequently distorting or attenuating the reading of the source/electrophysiology (collected at the electrode contact), a phenomenon described herein as “loading effect.”

The stimulation decoupler stage 156 can prevent a loading effect on the recording amplifier 112 inputs. As shown with in FIG. 2A, the stimulation decoupler 156 stage can be implemented with two antiparallel diodes 232. The antiparallel diodes 232 can decouple the stimulator 114 from being in parallel with the recording amplifier 112 (e.g., when the output of the stimulator is 0 mA or 0V), as often the stimulator output impedance is much lower (than the recording amplifier).

The current limiter stage 154 can limit the current that can flow to the recording amplifier (e.g., the current output to the “to Rec” port 160). As a non-limiting example, the current limiter stage 154 can be implemented with a current limiting diode or current limiting diode equivalent. A non-limiting example of a current limiting diode equivalent is a JFET 212 and a resistor 214. The current limiter stage 154 can tune the current being limited.

FIG. 2B illustrates a recording and stimulation setup 250 for stimulation and recording in humans including the FPClipre device 110 described with reference to FIG. 2A. Reference 164 and ground 166 are connected to scalp needles on the patient's head. The recording amplifier 112 and stimulator 114 can be the same or separate devices. The stimulator 114 return electrode/pad 270 can be attached to the body of the patient (e.g., the patient's shoulder).

The implementation of the present disclosure illustrated in FIG. 2B can enable recording and stimulation from the same electrode 102 using passive stages (e.g., the current limiter stage 154, voltage range limiter stage 158 and stimulator decoupler stage 156) illustrated in FIG. 2A. The implementation shown in FIG. 2B can perform recording and stimulation from the same electrode 102, even in challenging setups/configurations (due to stimulation artifacts) such as monopolar recordings and monopolar stimulation. In some implementations recovery of electrophysiology from every contact of a DBS lead can be of importance to enable the highest spatial localization of the biomarker of interest on the lead geometry. The implementation illustrated in FIG. 2B can be used in other scenarios (aside from DBS) to recover electrophysiology from the same electrode 102 contact that is being stimulated.

FIGS. 3A and 3B illustrate a comparison of an implementation of a Y-splitter-based implementation with an implementation including an FPClipre.

FIG. 3A illustrates a diagram of the components from the recording/stimulating setup modeling including a Y splitter 334, where the stimulator and recording amplifier are connected to the patient through a Y-connector. The implementation including the Y-splitter 334 shown in FIG. 3A can require a high dynamic range to limit saturation and consequently ADCs with higher resolution. This can be undesirable, particularly in the context of embedded devices (such as DBS neurostimulators). Furthermore, protection clipping stages 310 (typically antiparallel clipping diodes) in the recording amplifier 312 used to limit over-voltage at the differential inputs of the recording amplifier can interfere with the delivery of stimulation by limiting the amount of current that can be delivered to the patient. This can occur because the clipping diodes/stage 310 illustrated in FIGS. 3A and 3B can become conductive while delivering therapeutic stimulation through electrode(s), which consequently can cause the clipping diodes 310 to sink part of the current that was supposed to be delivered to the patient 330 through the recording amplifier 112. A non-limiting example scenario where this can happen is when a DBS lead with a contact impedance of 3 kOhm is stimulated with 5 mA current, leading to 15V at the amplifier stage, which would be above the forward voltage of the protection clipping diodes/stage, and therefore current would be sunk by the amplifier stage itself (through the clipping diodes/stage), and not the patient's 330 electrode. An implementation of an FPClipre 110 can allow both the recording and stimulation from the same contact, without incurring the downsides of the Y-splitter 334 setup illustrated in FIG. 3A.

FIG. 3B illustrates a diagram of the components from the recording/stimulating setup including the FPClipre instrumentation according to an implementation of the present disclosure. As shown in FIG. 3B, the stimulator 114 and recording amplifier 112 are connected to the patient through the FPClipre 110. FIG. 3C illustrates a simulation of the device illustrated in FIG. 3A, and shows how the antiparallel clipping diodes within the recording amplifier 112 (Vf=4.4V at 10 mA) can cause inadvertent stimulation current sinking into the recording amplifier instead of the patient, thus being unable to deliver the desired stimulation current to the patient, and potentially causing damage to the recording instrumentation. FIG. 3D illustrates a simulation of the example implementation of the present disclosure shown in FIG. 3B and shows that the current delivered by the stimulator is almost fully applied to the patient. The example implementation can limit the current delivered to the recording amplifier, while allowing simultaneous recording and stimulation. In this configuration the maximum current loss was below 20 uA (light shaded line), as per the design and acceptable considering the typical stimulation range (<0.67% loss for 2 mA, <0.40% loss for 5 mA).

In some devices, two elements (e.g., a stimulator and recording amplifier) are connected through a Y-splitter 334 to the patient electrode 102, as illustrated in FIG. 3E. Alternatively, devices can include a switching stage 370, as illustrated in FIG. 3F, that can enable the connection to the patient electrode 102 to either the recording amplifier or to the stimulator.

FIGS. 3E-3F illustrate additional examples of implementations including a Y-splitter 334 and a switching stage 370. In the implementation with a Y-splitter 334, the patient electrode 102 contact can be connected at the same time to both the stimulator 114 and recording amplifier 112, and consequently, there can be a large stimulation artifact at the recording stage, but consequently no switching artifact. However, saturation of the recording amplifier 112 can result in longer recovery times (e.g. ˜15 ms). Consequently, the recording amplifiers 112 used in a Y-splitter configuration usually have a wide dynamic range (e.g. 1-2V), to avoid saturation as much as possible, but have the downside of requiring higher complexity in the amplifier design to keep a low signal to noise ratio (SNR), and a higher analog to digital converter (ADC) resolution (e.g. 24 bit) to have sufficient bit resolution to sample the electrophysiology of interest (<1 μV). Furthermore, as shown in FIG. 3E antiparallel clipping diodes 310 can be included in the recording amplifier 112 to prevent over-voltage of the inputs. However, antiparallel clipping diodes 310 in the recording amplifier 112 can interfere with the delivery of stimulation and can limit the amount of current that is possible to deliver to the patient.

In the example implementations shown in FIG. 3E and 3F, reference and ground are connected to scalp needles on the patient head. The stimulator return electrode/pad 270 is attached to the patient shoulder. I/O ports are: “from Pat” connected to contact 1 from the patient electrode; “from Stim” connected to the stimulator output; “to Rec” connected to the recording amplifier input; “REF” connected to the reference of the patient and recording amplifier. In the example implementation shown in FIG. 3E, the patient electrode is connected to both stimulator and recording instrumentation through a Y-split connector. In the example implementation shown in FIG. 3F, the patient electrode is connected either to the stimulation or the recording amplifier instrumentation through a switch. This configuration does not allow to record data from the patient electrode during stimulation as contact 1 is physically disconnected from the recording amplifier.

FIG. 3F illustrates an implementation including a switching stage 370. The switching stage can keep the stimulating electrode contact connected to the stimulator for the whole duration of the stimulation, consequently not allowing the recording of electrophysiology from that contact, as there would be no physical connection to the recording amplifier. Therefore, implementations of the present disclosure including an FPClipre device can enable simultaneous stimulation and recording that is not possible in a switch-based implementation where the stimulating contact is connected to the stimulator for the whole duration of the stimulation. In some switch-based implementations, such as the one illustrated in FIG. 3F, the switching stage 370 can quickly switch from stimulation to recording after delivering each pulse. This switching can add design complexity by requiring tightly coupled stimulator and switching stages 370, and can be prone to mechanical/hardware failure in the short term. Variations of this implementation can require an active stage that switches (completely or partially) between stimulation and recording instrumentation. This, however, can still lead to large artifacts and limit recording configurations to bipolar montages or specific stimulation configurations (bipolar recording from symmetrical contact locations to the stimulating contact, monopolar stimulation. Accordingly, implementations of systems including the FPClipre illustrated with respect to FIGS. 1A-2B and 3B, can be simpler to design and construct than systems including switching stages 370.

Implementations of the present disclosure can enable stimulation and recording from the same electrode without using a switching-based setup. Switching based setups (illustrated in FIG. 3F) can require active methodologies (such as fast-switching relays) which can add complexity to the stimulation/recording setup, and can require a logic/processing unit to tightly control the fast actuation of the switching stage 370, and can also require communication between the stimulator and switching stage 370. Additionally, fast switching relays (e.g., reed relays) can be highly prone to failure in the short term (e.g. with chronic experiment using continuous 130 Hz stimulation). In contrast, implementations of the present disclosure can clip the signal only during the stimulation pulse delivery using the voltage limiter stage 158 and the current limiter stage 154, as shown in FIG. 1B. By clipping the signal during stimulation pulse delivery, implementations of the present disclosure can avoid saturation of the recording amplifier any allow simultaneous recording and stimulation from the same contact. Additionally, implementations of the present disclosure can be energy efficient and can operate without a separate logic/processor unit, and can operate without a driver or other form of active synching. Accordingly, implementations of the present disclosure can be suitable for integration or embedding in existing commercial amplifiers and stimulation setups. (e.g., as the FPClipre stage 110 illustrated in FIG. 2B). Additional example applications for implementations described herein include implantable devices such as DBS neurostimulators.

### EXAMPLES

An example implementation of the FPClipre device was studied. The example implementation of the FPClipre was tested in an in-vitro setup to simulate the typical clinical recording/stimulating setup used during DBS surgeries.

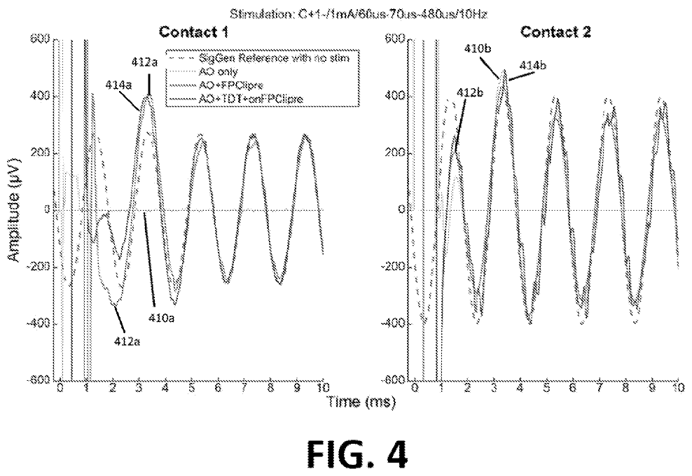

With reference to FIG. 4, the example implementation showed viable recovery of the injected test signal during stimulation, which was not possible when using only the clinical commercial device sold under the trademark Neuro Omega by Alpha Omega. In the example results illustrated in FIG. 4, the NeuroOmega from Alpha Omega (AO) was used as stimulator and configured for monopolar stimulation to contact 1, C+1-, at 1 mA, with bipolar asymmetric pulse (60 vs 480 us), with 70 us interpulse interval. Two recording systems were used: AO and Tucker-Davis Technologies Labrat (TDT). Recorded signals from two contacts are presented: contact 1 which was also the stimulating contact 1 and contact 2 (adjacent contact on the same DBS electrode). Data was recorded within saline solution. The testing signal to be recovered was a sinusoid at 500 Hz. Data were detrended to remove slow artifactual trend due to stimulation artifact and electrode-water impendance. “SigGen Reference” was recorded with AO and with no stimulation. “AO only” was recorded with AO, and shows that no signal can be recovered from contact 1, as it is connected to the stimulation stage. “AO+FPClipre” 412a 412b was recorded while the proposed method and system were connected to AO for both data acquisition and stimulation. The test signal is now recoverable from the stimulating contact as well. “AO+TDT+FPClipre” 414a 414b was recorded while the proposed method and system were connected to TDT amplifier for data acquisition, while AO was used as stimulator. This example shows that implementations of the present disclosure can link separate stimulation and amplifier stages without Fan active stage for switching.

The signal recovered by the Neuro Omega only is shown as 410a and 410b in the plots 400a 400b for contacts 1 and 2, respectively.

Still with reference to FIG. 4, the signal recovered using the Neuro Omega with the FPClipre is illustrated as curve 412a 412b, and the signal recovered using the Neuro Omega, FPClipre, and a separate recording amplifier, sold under the trademark Labrat by Tucker-Davis Technologies, is illustrated by curves 414a 414b.

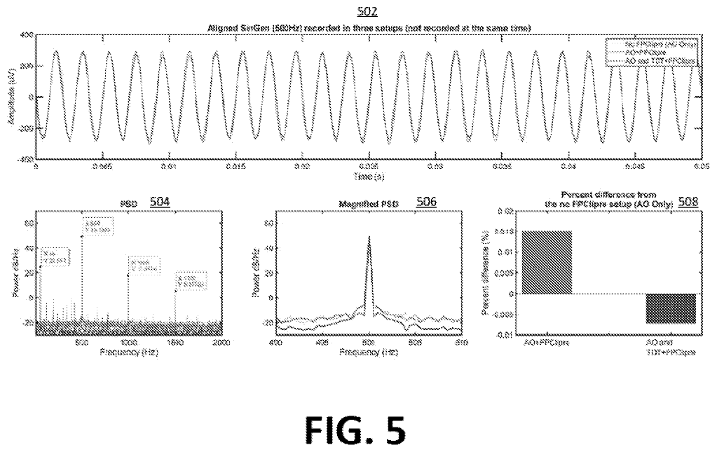

As shown in FIG. 5, the example implementation demonstrated excellent signal fidelity with a negligible difference from the reference testing signal <0.02%.

FIG. 5 shows a time domain test signal 502 recorded with and without FPClipre using the same device that is used for stimulation (the Neuro Omega), or a different amplifier (Tucker-Davis Technologies). The power spectral density 504 and magnified power spectral density 506 for the same signals is shown in the frequency domain. A plot 508 of percent difference of the recorded test signal (SigGen) power (at 500 Hz, BW=1 Hz) when FPClipre is used is less than 0.02% is also illustrated, demonstrating excellent signal fidelity.

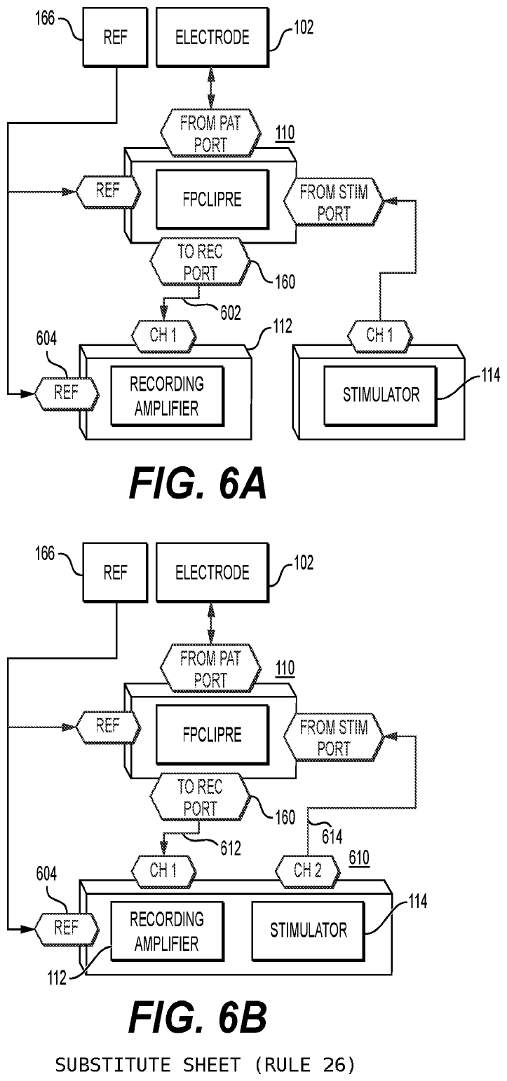

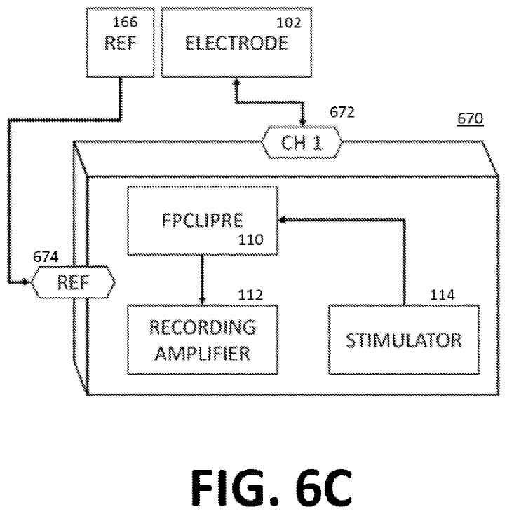

According to some implementations of the present disclosure, the FPClipre can also be used as add-on with a separate recording amplifier and stimulator, as shown in FIG. 6A. The FPClipre 110 can be used with a separate recording amplifier 112 and stimulator 114 in the configuration shown in FIG. 6A. In this implementation, the recording amplifier input channel 602 can be connected to the FPClipre “to Rec” port 160, the reference (“REF”) to both the FPClipre “REF” port 164 and the recording amplifier “REF” port 604. The stimulator output channel can be connected to the “from Stim” port 162 of the FPClipre 110, and the electrode 102 can be connected the FPClipre 110 “from Pat” port 152. This can enable recording from the same electrode used for stimulation, which in this use case can be collected by “CH 1” 602 of the recording amplifier 112.

According to some implementations of the present disclosure, the FPClipre 110 can be used as add-on with a combined recording amplifier and stimulator system 610. The FPClipre 110 can be used when there is a system with a combined and inseparable recording amplifier and stimulator, as shown by the configuration illustrated in FIG. 6B. This configuration can use two channels 612 614 to record/stimulate for one electrode. The two channels can be referred to as “CH 1” 612 and “CH 2” 614. The “CH1” 612 output of the recording/stimulating system 610 can be connected to the FPClipre 110 “to Rec” port 160, the reference (“REF”) can be connected to both the FPClipre “REF” port and the recording/stimulating system “REF”, the recording/stimulating system “CH2” can be connected to the FPClipre “from Stim” port 162, and the electrode 102 can be connected to the “from Pat” port 152. This can enable recording from the same electrode 102 used for stimulation, which in this use case can be collected by “CH 1” 612 of the combined recording amplifier and stimulator system 610. In some implementations, “CH 2” 614 can be used only for stimulation purposes, and not for collecting viable electrophysiology.

As shown in FIG. 6C, in some implementations of the present disclosure, FPClipre 110 can be integrated and combined with a recording amplifier and stimulator system 670. The FPClipre 110 can be directly integrated with a recording amplifier 112 and stimulator 114 as a single system package 670. The combined recording amplifier and stimulator system 670 can be modeled like the configuration illustrated in FIG. 6A and can include the same functionality. Similarly to FIG. 6A, an operator of the implementation shown in FIG. 6C is only provided with connections to the “CH1” port 672 and “REF” 674 port. The reference (“REF”) 166 can be connected to the combined system “REF” port, and the electrode to the combined system “CH 1” port. This can enable recording from the same electrode used for stimulation, which in this example implementation is illustrated as “CH 1”.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A device for recording a signal at a stimulating electrode, the device comprising:
a stimulator port operably connected to a stimulator decoupler stage;
an input port operably connected to the stimulator decoupler stage and a current limiter stage, wherein the current limiter stage is configured to limit a current flowing from the input port to a recording port and wherein the stimulator decoupler stage is configured to prevent a loading effect on an input port of a recording amplifier; and
a voltage range limiter stage operably connected to the current limiter stage and the recording port and configured to limit a voltage output to a predetermined recording amplifier range.

2. The device of claim 1, wherein the stimulator decoupler stage comprises a pair of antiparallel diodes connected in parallel.

3. The device of claim 1, wherein the stimulator decoupler stage comprises a plurality of antiparallel diodes connected in series.

4. The device of claim 1, wherein the current limiter stage comprises a Junction Field Effect (JFET) constant current source, wherein the JFET constant current source comprises a JFET transistor and a resistor.

5. The device of claim 4, wherein the current limiter stage comprises a plurality of JFET constant current sources.

6. The device of claim , wherein the voltage range limiter stage comprises a pair of antiparallel diodes connected in parallel.

7. The device of claim 1, wherein the device further comprises a ground port, and the voltage range limiter stage is operably connected between the ground port and the recording port.

8. The device of claim 1, wherein the device is configured to receive an electrophysiology signal.

9. The device of claim 1, wherein the device is configured amplify neural activity during deep brain stimulation.

10. A system for simultaneously stimulating and recording data using an electrode, the system comprising:
a stimulator port operably connected to a stimulator decoupler stage;
a ground port;
an input port operably connected to the stimulator decoupler stage and a current limiter stage, wherein the current limiter stage is configured to limit a current flowing from the input port to a recording port and wherein the stimulator decoupler stage is configured to prevent a loading effect on an input port of a recording amplifier wherein the recording amplifier is operably connected to the recording port and the ground port;
a probe comprising one or more electrodes operably connected to the input port;
a voltage range limiter stage operably connected to the current limiter stage and the recording port and configured to limit a voltage output to a predetermined recording amplifier range; and
a stimulator operably connected to the stimulator port.

11. The system of claim 10, wherein the stimulator decoupler stage comprises a pair of antiparallel diodes connected in parallel.

12. The system of claim 11, wherein the stimulator decoupler stage comprises a first pair of antiparallel diodes that are connected in parallel, and a second pair of antiparallel diodes connected in parallel, and wherein the first and second pair of antiparallel diodes are connected in series.

13. The system of claim 10, wherein the current limiter stage comprises a JFET constant current source, wherein the JFET constant current source comprises a transistor and a resistor.

14. The system of claim 10, wherein the current limiter stage comprises a plurality of JFET constant current sources.

15. The system of claim 10, wherein the voltage range limiter stage comprises a pair of antiparallel diodes connected in parallel.

16. The system of claim 10, wherein the system further comprises a ground port, and the voltage range limiter stage is operably connected between the ground port and the recording port.

17. The system of claim 10, wherein the stimulator and recording amplifier are a combined stimulator and recording amplifier.

18. The system of claim 10 wherein the stimulator, recording amplifier, voltage range limiter stage, current limiter stage, and stimulator decoupler stage are part of a single device.

19. The system of claim 10, wherein the system is configured to receive an electrophysiology signal.

20. The system of claim 10, wherein the system is configured amplify neural activity during deep brain stimulation.
